(19) United States
(12) Patent Application Publication
(10) Pub. No.: US 2020/0342969 A1
(43) Pub. Date: Oct. 29, 2020

(54) MONITORING TREATMENT COMPLIANCE USING EVIDENCE-BASED GUIDELINES

(71) Applicant: Gallagher Bassett Services, Inc., Itasca, IL (US)

(72) Inventors: Jeffrey Austin White, Lighthouse Point, FL (US); Gary T. Anderberg, New Hope, PA (US)

(21) Appl. No.: 16/854,454

(22) Filed: Apr. 21, 2020

Related U.S. Application Data
(60) Provisional application No. 62/838,483, filed on Apr. 25, 2019.

(51) Int. Cl. G16H 20/00; G16H 10/60; G06Q 40/08; G06Q 30/02

(52) U.S. Cl. CPC G16H 20/00; G06Q 30/0206; G06Q 40/08; G16H 10/60

(57) ABSTRACT

A method includes receiving medical data records and computing a compliance score from an ideal treatment plan and an actual treatment, the actual treatment stored in medical data records. The method also includes determining whether the compliance score is greater than a compliance threshold and, in response to a determination that the compliance score is greater than the compliance threshold, storing a pass flag with a data record of the actual treatment. The method also includes, in response to a determination that the compliance score is less than the compliance threshold storing a fail flag with the data record of the actual treatment and controlling a graphical user display to indicate the data record includes the fail flag.

### CROSS-REFERENCES TO RELATED APPLICATIONS

This patent application claims priority to U.S. Provisional Patent Application Ser. No. 62/838,483 filed Apr. 25, 2019, which is incorporated herein by reference in its entirety.

### BACKGROUND

There is a continuing need to improve on health care outcomes. The growing complexity of today's medicine, is characterized by more: more to know, more to do, more to control, more to watch, and more people involved. This need for “more” makes medical insurance claims more difficult to manage. Faced with these rapid changes, the insurance industry has lagged in its ability to optimize medical care to improve claim outcomes. Consequently, medical management strategies have not evolved beyond yesterday's elementary cost driven, not care driven measures, such as the implementation of PPO networks, fee schedules and retrospective reviews.

### SUMMARY

An aspect of the disclosed embodiments includes a system that includes a processor and a memory component. The memory component having medical data records and instructions stored thereon, when executed by the processor, causes the processor to: compute a compliance score from an ideal treatment plan and an actual treatment, the actual treatment being stored in the medical data records; determine whether the compliance score is greater than a compliance threshold; in response to a determination that the compliance score is greater than the compliance threshold, store a pass flag with a data record of the actual treatment; and in response to a determination that the compliance score is less than the compliance threshold store a fail flag with the data record of the actual treatment and control a graphical user display to indicate the data record includes the fail flag.

Another aspect of the disclosed embodiments includes a method that includes receiving medical data records and computing a compliance score from an ideal treatment plan and an actual treatment, the actual treatment stored in medical data records. The method also includes determining whether the compliance score is greater than a compliance threshold and, in response to a determination that the compliance score is greater than the compliance threshold, storing a pass flag with a data record of the actual treatment. The method also includes, in response to a determination that the compliance score is less than the compliance threshold storing a fail flag with the data record of the actual treatment and controlling a graphical user display to indicate the data record includes the fail flag.

Another aspect of the disclosed embodiments includes a system that includes a processor and a memory component. The memory component having medical data records and instructions stored thereon, when executed by the processor, causes the processor to: receive an ideal treatment plan corresponding to an injury of an individual; identify an actual treatment in the medical data records corresponding to the ideal treatment plan; generate, using a machine learning system, a compliance score based on the ideal treatment plan and the actual treatment; determine whether the compliance score is greater than a compliance threshold; in response to a determination that the compliance score is greater than the compliance threshold store a pass flag with a data record of the actual treatment and control a graphical user display to indicate the data record includes the pass flag; and in response to a determination that the compliance score is less than the compliance threshold store a fail flag with the data record of the actual treatment and control the graphical user display to indicate the data record includes the fail flag.

These and other aspects of the present disclosure are disclosed in the following detailed description of the embodiments, the appended claims, and the accompanying figures.

### BRIEF DESCRIPTION OF THE DRAWINGS

In the drawings, which are not necessarily drawn to scale, like numerals may describe similar components in different views. Like numerals having different letter suffixes may represent different instances of similar components. Some embodiments are illustrated by way of example, and not limitation, in the figures of the accompanying drawings in which:

FIG. 1 generally illustrates a block diagram showing an example system according to the principles of the present disclosure.

FIG. 2 generally illustrates block diagram illustrating further details of the system according to principles of the present disclosure.

FIG. 3 generally illustrates a schematic diagram illustrating data, which may be stored in the database of the system, according to principles of the present disclosure.

FIG. 4 generally illustrates a schematic chart for insurance claims flow according to principles of the present disclosure.

FIG. 5 generally illustrates a flow diagram of a method according to principles of the present disclosure.

FIG. 6 generally illustrates a flow diagram of a method according to various aspects of the present disclosure.

FIG. 7 generally illustrates a flow diagram according to various aspects of the present disclosure.

FIG. 8 generally illustrates a block diagram illustrating a representative software architecture, which may be used in conjunction with various hardware architectures herein described.

FIG. 9 generally illustrates a block diagram illustrating components of a machine, according to the principles of the present disclosure, able to read instructions from a machine-readable medium (e.g., a machine-readable storage medium) and perform any one or more of the methodologies discussed herein.

FIGS. 10A and 10B generally illustrate a display showing a graphical user interface including a compliance score according to various aspects of the present disclosure.

FIGS. 11A and 11B generally illustrate a display showing a graphical user interface including a compliance score according to various aspects of the present disclosure.

FIGS. 12A and 12B generally illustrate a display showing a graphical user interface including a compliance score applied on a provider basis according to various aspects of the present disclosure.

FIGS. 13A and 13B generally illustrate a display showing a graphical user interface including a compliance score applied on a provider basis according to various aspects of the present disclosure.

FIG. 14 generally illustrates a display showing a graphical user interface including a compliance score applied on a provider and geographic basis according to various aspects of the present disclosure.

FIG. 15 is a flow diagram generally illustrating a method according to various aspects of the present disclosure.

### DETAILED DESCRIPTION

The description that follows includes systems, methods, techniques, instruction sequences, and computing machine program products that embody illustrative embodiments of the disclosure. In the following description, for the purposes of explanation, numerous specific details are set forth in order to provide an understanding of various embodiments of the inventive subject matter. It will be evident, however, to those skilled in the art, that embodiments of the inventive subject matter may be practiced without these specific details. In general, well-known instruction instances, protocols, structures, and techniques are not necessarily shown in detail.

Among other things, embodiments of the present disclosure improve the functionality of treatment care methods and systems. An organization can provide ways to improve treatment outcomes by holding patients and caregivers to evidence-based medical guidelines.

Embodiments described herein relate to a method and a system for measuring the quality of care delivered by medical providers to injured claimants. Evidence-based medicine (EBM) guidelines can be mapped to medical billing records to derive a compliance score. The compliance score is used as a proxy to indicate the relative level of care that falls within the treatment guidelines established by a predetermined threshold of acceptability for quality assurance purposes. Clinical research has demonstrated that improved outcomes for medical treatment and on claims related to the medical treatment can be driven in association with a predominance of medical treatments that match the EBM treatment guidelines. The compliance score can be used to monitor adherence to the treatment guidelines and quality of medical care at both the medical provider and claim level. When the compliance score falls below the critical threshold, containing an unacceptable level of inappropriate or unnecessary medical care, a series of workflows and alerts are triggered to draw attention to a claim or provider for consideration of corrective actions. That is, the compliance score related to any individual treatment regime can flag a particular treatment or course of treatments for further investigation.

In some embodiments, the compliance score includes a synthetic metric (e.g., a value that is a result of a combination of other natural metrics). For example, the compliance score may include a signal index, which encompasses a number of primary measurements in a single integer (e.g., referred to as a synthetic metric). The compliance score, as described herein, may be a result of a combination of a plurality of natural metrics, which are typically collected in typical medical claims processes. Accordingly, in some embodiments, the generating the compliance score can be performed using existing metric data (e.g., without having to generate additional data).

Embodiments described herein further provide interactive graphical user interfaces to use compliance scores. Such graphical user interfaces may include dashboards with drill down capability at the claim level and provider level to support rapid identification of trends and outliers. The dashboards provide tables, charts, and color-coded items to draw attention to poor performance or high performance situations, e.g., as judged by the compliance score in an example. The dashboards can be used for quality assurance purposes or for purposes of monitoring program performance.

In some embodiments, the compliance scores may be calibrated, using a machine learning system trained using a large database, such as those described herein. The database stores all aspects of the individual claim, in addition to treatment components stored in a database used to generate the compliance score. In order to model probable claim outcomes by using machine learning against a unique history (e.g., most recent ten year claim history) of all corresponding insurance claims, including workers' compensation claims, accident claims, illness claims, treatment claims and the like. Such calibration of the compliance scores may be a function of a large data modelling application to determine compliance score values that represent acceptable medical practice of medical providers and compliance score values that represent non-acceptable practice of medical providers (e.g., within the context of the probable outcomes predicted for each individual claim). The calibration of the compliance scores may be non-arbitrary and may be based on differential rates of meaningful clinical events and/or other relevant claim characteristics across a broad array of medical caregivers or professionals over time.

In some embodiments, the compliance scores may be used in the management of insurance claims, such as workers' compensation claims and/or in the management of claims in other medical claims. In some embodiments, the compliance scores may be calibrated in according to a first calibration protocol. The compliance scores, when calibrated according to the first calibration protocol, may be used to trigger interventions regarding individual claims. Additionally, or alternatively, the compliance scores may be calibrated according to a second calibration protocol, different from the first calibration protocol. The compliance scores, when calibrated according to the second calibration protocol, may be used to monitor provider performance and/or retention/non-retention decisions in the management of large medical services networks. It should be understood that the compliance scores may be calibrated in any suitable manner according to any calibration protocols including those described herein and/or other calibration protocols.

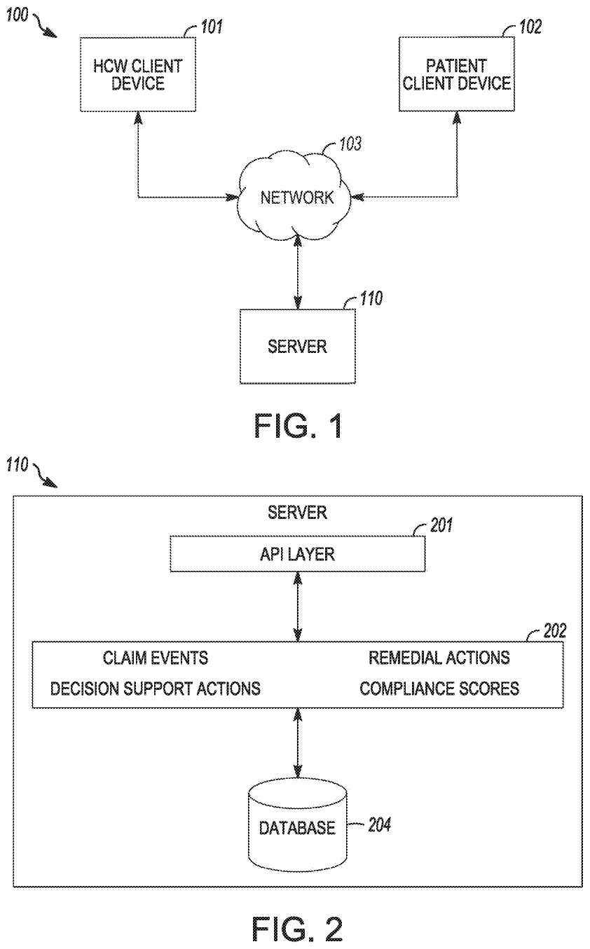

FIG. 1 generally illustrates a block diagram showing a system 100 according to the principles of the present disclosure. The system 100 can be a medical care service system that includes a server 110, a health care worker (HCW) client device 101, a patient-related client device 102 that are communicatively coupled over a network 103 (e.g., Internet, telephony network). While FIG. 1 illustrates a single HCW client device 101 and a single patient-related client device 102, it is understood that a plurality of HCW client devices 101 and a plurality of patient-related client devices 102 can be included in the system 100 in other embodiments. As used herein, the term “client device” may refer to any machine that interfaces to a communications network (such as network 103) to obtain resources from one or more server systems or other client devices. A client device may be, but is not limited to, a mobile phone, desktop computer, laptop, portable digital assistants (PDAs), smart phones, a wearable device (e.g., a smart watch), tablets, ultrabooks, netbooks, laptops, or any other communication device that a user may use to access a network.

The network 103 may include, or operate in conjunction with, an ad hoc network, an intranet, an extranet, a virtual private network (VPN), a local area network (LAN), a wireless network, a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network and the coupling may be a Code Division Multiple Access (CDMA) connection, a Global System for Mobile communications (GSM) connection, or other type of cellular or wireless coupling. In this example, the coupling may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, fifth generation networks, Universal Mobile Telecommunications System (UMTS), High Speed Packet Access (HSPA), Worldwide Interoperability for Microwave Access (WiMAX), Long Term Evolution (LTE) standard, others defined by various standard setting organizations, other long range protocols, or other data transfer technology.

In the example shown in FIG. 1, a user using the patient-related client device 102 can establish a communication session with an HCW associated with the HCW client device 101. The HCW agent can be a human agent or an automated agent, e.g., on behalf of an organization that tracks and administers health related information and outcomes. The automated agent can be an interactive voice response (IVR), a virtual online assistant, or a chatbot. During a communication session between the user and the agent, the server 110 stores data relating to an outcome, a treatment event, or other healthcare related data.

FIG. 2 shows the server 110 that includes an application program interface (API) layer 201 to interact with outside data sources and the devices 101, 102 and a processing system 202 in communication with both the API layer 201 and the database 204. The server 110 can operate determine compliance scores using an ideal treatment derived from evidence based medicine treatment guidelines and the actual treatment received by a person, e.g., a worker in a worker's compensation claim or any other suitable insurance or treatment claim. The computed compliance score represents the divergence of the actual treatment from the ideal treatment for a given injury or illness. The compliance score can be flagged in a data record and the flags displayed in a graphical user interface. The compliance score provides the opportunity to rate health care workers and clinics. The compliance scores can be analyzed by geographic area when associated with patient treatment records, health records, health care provider records, health clinic records, and the like.

The API layer 201 in the server 110 can include a server structure including a processor operably coupled to a memory. The API layer 201 is coupled to, and provides a programmatic interface to, outside data sources, e.g., electronic devices and the processing system 202. For example, the processing system 202, using the API server 201, can access clinical health data, medical treatment guidelines, medical best practices, to develop evidence based medicine (EBM) guidelines. The processing system 202, using the API layer 201, can access medical treatment for an individual, e.g., from database 204 or from devices 101, 102. The system 202 can access medical billing records to derive treatment of the individual. The system 202 can develop or access claim events from the billing records. Claim events can be injuries, which can trigger a worker's compensation claim or other insurance claim for medical injuries. Claim events can be illnesses. The claim events can also include medical visits, treatment events, surgery, prescriptions, physical therapy, opioid counseling, and the like deriving definitions from International Classifications of Diseases (ICD) and Current Procedural Terminology (CPT) medical coding system. The system 202 can develop or access remedial actions, including but not limited to, three point contact, evaluations, investigations, notes from medical providers, check-ins, medical care provider visit reminders, status checks from medical providers, direct contacts with the injured worker and the like. The system 202 can also develop novel decision support actions that use analysis to improve outcomes. The decision support actions can include provider compliance scores, automated guideline compliance checks, expert review, nurse review, peer review and the like. It may also include automated messages to a claimants mobile app, e.g., on patient client device 102, recommending actions or information. The database 204 can store all of this data used by the system 202.

The system 202 can output processed data through the API layer 201 to external devices to produce a graphical user interface on an electronic display.

The system 202 can measure the quality of care delivered by medical providers to injured claimants using a compliance score. The compliance score can include a measure of the quality of treatment. The quality of treatment can be based how closely the treatment received by a patient meets a standard of care, e.g., a standard of care developed from evidence based medicine (EBM). The EBM can be formed from scientific medical research on outcomes to produce a guideline for a specific injury or illness. The evidence based medicine (EBM) guideline based on analysis of medical billing records. The compliance score may be a proxy indicative of the relative level of care that falls within the EBM treatment guidelines established by a predetermined threshold of acceptability for quality assurance purposes. It has been shown that improved outcomes on claims is found in association with a predominance of medical treatments that match the EBM treatment guidelines. The compliance score (CS) can be used to monitor adherence to the EBM treatment guidelines and quality of medical care at both the medical provider and claim level. When the compliance score falls below the critical threshold, containing an unacceptable level of inappropriate or unnecessary medical care, a series of workflows and alerts can be triggered to draw attention to a claim or provider for consideration of corrective actions.

The API layer 201 receives data (e.g., claim event data, remedial actions taken, and the like) and transmits data (e.g., decision support actions triggers, compliance score, and the like) between the agent client device 101 and the system 202. The API layer 201 can receive and transmit data between the agent client device 101 and the system 202 in real-time. Specifically, the API layer 201 provides a set of interfaces (e.g., routines and protocols) that can be called or queried by the system 202 in order to invoke functionality of the triggered by the compliance score.

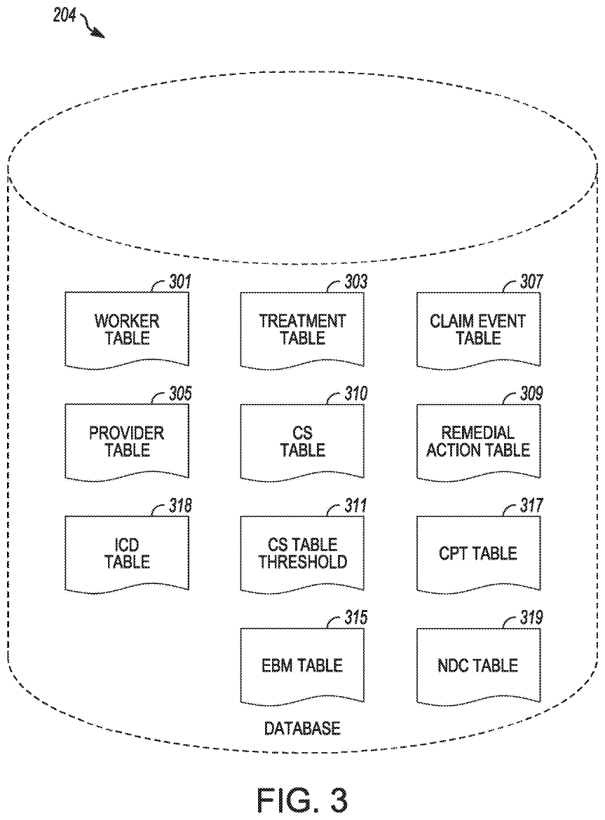

FIG. 3 generally illustrates a schematic diagram 300 illustrating data that is stored in the database 204 of the system, according to the principles of the present disclosure. While the content of the database 204 is shown to comprise a number of tables, the data could be stored in other types of data structures (e.g., as an object-oriented database). The database 204 includes an injured worker (e.g., patient) table 301, a treatment table 303, a provider table 305, a claim event table 307, and a remedial action table 309. The injured worker table 301 stored data relating to the injured worker, e.g., identifying information, diagnosis, doctor, other medical providers, medical treatment, dates of injury and treatment, individual compliance score, which can be derived from the diagnosis and treatment being performed. The treatment table 303 can include types of treatments that relate to a diagnosis. The provider table 305 can include identification information for an individual provider, their practice, medical specialty, historical treatments in total, specific treatments for individuals, and billing information. The claim event table 305 can store all events that relate to an individual or an injury to an individual. The remedial action table 307 can store all remedial actions taken for a claim and the results therefrom.

The database 204 can also store data relating to the compliance score, e.g., a compliance score table 310 and a compliance score threshold table 311. The compliance score table 310 can store the compliance score on a per injury basis, e.g., a compliance score value is assigned to any triggering claim event, i.e., an injury to a worker. The compliance score value is a compliance score derived from the analysis of best practices. The best practices are derived from historical outcomes and medical research and literature. The compliance score threshold table 311 stores the threshold at which an individual's compliance score will trigger remedial action. An individual's compliance score is calculated based on the diagnosis and treatments/care provided which can be stored in the patient (injured worker) table 301. The individual's compliance score is compared to the threshold compliance score.

The database 204 can also store data relating medical treatment and classification, e.g., an Evidence Based Medicine Reference (EBM) table 315, the Current Procedural Terminology (CPT) table 317, the International Classifications of Diseases (ICD) code table 318, the National Drug Code (NDC) table 319. The EBM table 315 may include data corresponding to any suitable evidence based medicine guidelines or standards, such as Official Disability Workers Guideline data, Presley Reed Guideline data, American Medical Association Guideline data, other suitable guidelines or standards data, or a combination thereof. In some embodiments, the EBM table 315 includes data corresponding to two or more evidence based medicine guidelines or standards, including two or more of those described herein and/or any other suitable evidence based medicine guidelines or standards. The EBM table 315 stores information to determine, at the time of treatment, the frequency and extent of services presumed to be medically necessary and appropriate for compensable injuries. The CPT table 317 stores information to identify medical, surgical, and diagnostic services and is designed to communicate uniform information about medical services and procedures among physicians, coders, patients, accreditation organizations, and payers for administrative, financial, and analytical purposes. The International Classifications of Diseases (ICD) code table 318 stores information to identify diseases, disorders, symptoms, poisonings, adverse effects of drugs and chemicals, injuries and other reasons for patient encounters. Diagnostic coding in table 318 is the translation of written descriptions of diseases, illnesses and injuries into codes from a particular classification. In medical classification, diagnosis codes are used as part of the clinical coding process alongside intervention codes. Both diagnosis and intervention codes are assigned by a health professional trained in medical classification such as a clinical coder or Health Information Manager. The data in tables 315-319 can be used to calculate the compliance score for the individual and the desired compliance score in table 310. The threshold compliance score is set based on a deviation from the desired compliance score in table 310.

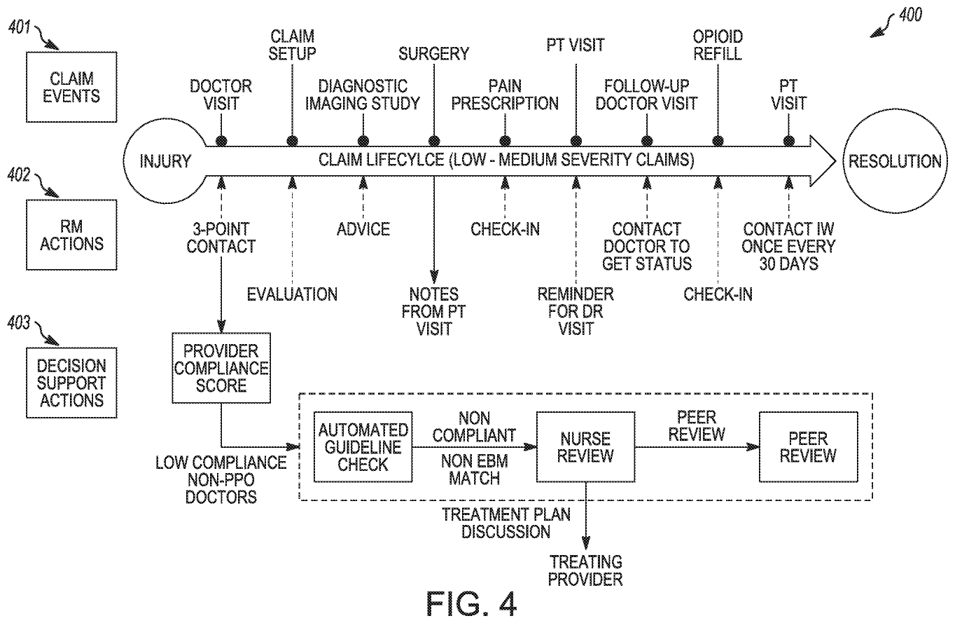

FIG. 4 is a schematic diagram of a system 400 of applying compliance score. The application of compliance score can be performed by the server 110 in FIG. 1. In one embodiment, a processor (e.g., circuitry dedicated to performing instructed tasks) included in the server 110 performs instructions, which can be stored in a memory, to implement compliance score from a triggering event (here shown as an injury) to an ending event (here shown as resolution). The system 400 can include a plurality of layers 401-403. The first layer 401 is the claim events layer, which includes events happening to the individual. The claim events can include a triggering event (e.g., an injury) followed by medical treatment. Medical treatment can include an initial visit to a medical care provider, a doctor visit and the like. A claim is set up, which can begin to populate the tables in the database. A treatment program is developed and can include imaging, surgery, prescriptions, pain treatment, physical therapy, follow-up doctor visits, prescription refills, additional medical care visits, other services, and a combination thereof.

The second layer 402 is the remedial actions layer, which includes actions taken to confirm various claim events and provide information to be used in compliance score. Remedial actions can include three-point contact (e.g., contacting the patient, the employer and the treating doctor). In an example, the three-point contact is the beginning of the process used in determining the nature and extent of injury and disability of workers' compensation claims and begins the claim process of returning the injured employee to work. Additional remedial actions include evaluation, advice, notes from physical therapy, check-in, doctor visit reminder(s), contact medical providers for status, additional check-ins, periodic contact of injured worker, and the like.

The third layer 403 is the decision support layer. A provider compliance score is calculated and if low compliance or below the compliance score threshold, additional decision support actions are performed. The additional decision support actions can include an automated guideline check. If the guidelines are not being followed or there is no evidence based medicine (EBM) guidelines, then a nurse can review the medical treatment to date. The nurse review can trigger a consultation with the treating provider to determine why the evidence based medicine (EBM) guidelines were not followed. The nurse review can also trigger a medical peer review of the treatment to date.

The remedial actions in remedial layer 402 and the decision support actions in layer 403 can be triggered when the compliance score for the individual falls below the compliance score for a given diagnosis or injury.

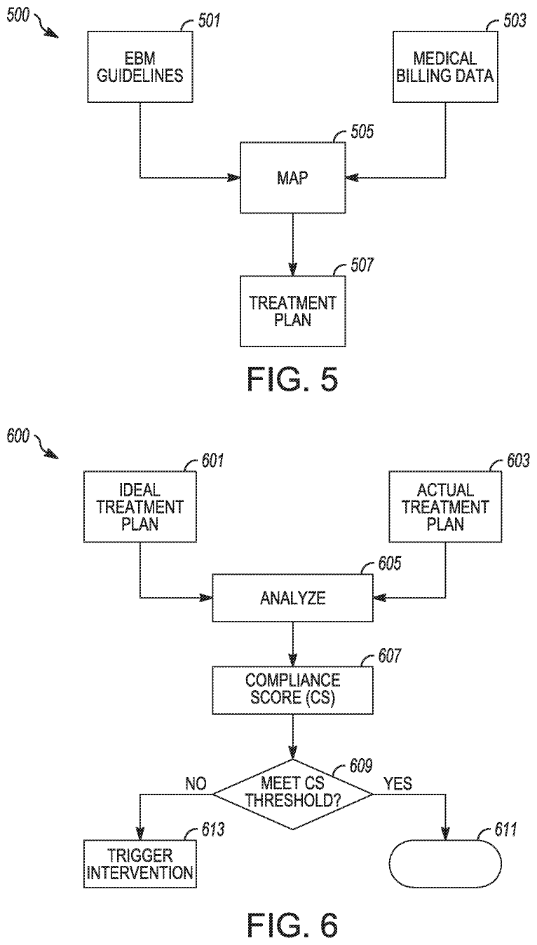

FIG. 5 is a flow diagram generally illustrating a method 500 of developing a treatment plan that can be used to create a compliance score baseline. The treatment plan can be based on evidence based medicine (EBM) guidelines and medical billing data.

At operation 501, the evidence-based medicine (EBM) guidelines are provided. The EBM guidelines are guidelines for each diagnosis or injury. The diagnosis can be a basis for a worker's compensation claim, if the injury occurred on the job. EBM guidelines are standardized treatment that have been codified and refined over decades by gathering, evaluating, and sharing battle-tested, proven clinical treatments. The process of developing medical practices based on the best available evidence from scientific research is a type of EBM. In the case of methods and systems to track, approve, and provide treatment, they can be optimized by accessing analyzing reams of medical data. In some cases the quantity of data in the database related to claims may exceed the amount of data that an individual person can process to develop the compliance score as discussed herein. In workers' compensation, the EBM guidelines are used as a yardstick to measuring the type, frequency, timing and duration of expected care. The intent of the EBM guidelines is to combine best practice protocols and optimal care pathways to avoid potentially harmful, inappropriate care for an injured worker. EBM guidelines can be used by claims professions to help them navigate highly variable local practices.

At operation 502, the medical billing data related to a diagnosis is provided. The medical billing data 503 is filtered from a large database, e.g., multiple millions of medical diagnosis and an order of magnitude or more of related treatments. The medical billing data can be a stored at an insurer, e.g., a workers' compensation insurer.

At operation 505, the EBM guidelines 501 are mapped to the medical billing data 503. The medical billing data 503 provides a look at numerous, tens of thousands or even millions, diagnose, e.g., injuries, illnesses, etc. The treatment plan represents the statistically best treatment for the given diagnosis.

At operation 507, the treatment plan for a given diagnosis is output from operation 505 and is based on the EBM guidelines and the medical billing data. The treatment plan can be stored in the database 204.

FIG. 6 generally illustrates a flow diagram a method 600 to develop a compliance score. At operation 601, the ideal treatment plan is provided for a given diagnosis. The ideal treatment plan is the treatment plan developed in method 500. At operation 603, the actual treatment plan being executed for an individual is provided. The actual treatment plan is directed to an individual for a specific diagnosis.

At operation 605, the actual treatment plan is analyzed relative to the ideal treatment plan and outputs the compliance score to operation 607. The compliance score represents the alignment of the actual treatment plan to the statistically produced ideal treatment plan. The compliance score can be the percentage of care out of the total treatment plan for a given injury that complies with the ideal treatment plan. The compliance score can represent coherence to EBM guidelines, e.g., as a percentage from 1 to 100 and applied at the claim or medical provider level and serves as a proxy for quality of care. Other values can be used in place of a percentage, for example, a score of one to ten, a ratio, a fraction, and the like.

At a basic level, the compliance score is calculated by dividing the total cost or number of treatment compliance with the evidence based guidelines by the total cost or number of all treatment on the claim or associated with a provider. The compliance score can also be reported as a normalized score from zero to one, with zero being no compliance and one being complete compliance. The analysis of the treatments 601, 603 can be performed on a basis of the patient to produce a patient compliance score, e.g., for a specific injury or illness or over the lifetime of the individual. In the alternative, the analysis of the treatments 601, 603 can be performed on a basis of the medical provider to produce a provider compliance score. The analysis of the treatments 601, 603 can be performed on a basis of the medical clinic to produce a medical clinic compliance score. The provider and clinic compliance score(s) can be computed over time for compliance with the EBM guidelines for which the provider or clinic treats patients. The provider and clinic compliance score(s) can be statistical analysis of all the patients being treated by the provider or at a clinic by multiple HCWs. The compliance score can also be reported on a continuous basis and be cumulative in nature to derive a historical compliance rating.

In some embodiments, the compliance score may be generated using a machine learning system, such as a neural network, or other suitable machine learning system, executing a machine learning algorithm. The neural network may complies a plurality of interconnected neurons or nodes configured to use various computational and mathematical models to perform data analysis and other suitable functions. The machine learning system may be trained using the data records described herein. The machine learning system may receive the ideal treatment plan and actual treatment plan. The machine learning system may generate, using a machine learning system, a compliance score using the ideal treatment plan and the actual treatment plan for a respect claim.

The analysis operation 605 can calculate the compliance score as a number of medical treatments compliant with EBM guidelines divided the total number of medical treatments for the diagnosis. This provides a lower sensitivity compliance score.

At operation 609, the compliance score is compared to a compliance threshold. If the compliance score meets the threshold, then the process 600 ends at 611. If the compliance score does not meet the threshold, then an intervention is triggered at operation 613. The intervention can be one or more of the remedial actions at level 402 (FIG. 4).

The method 600 can be repeatedly performed at various times. Calculation of the compliance on a regular basis for each medical provider can be used to evaluate their use of appropriate care. If expectations are not met, the provider can be flagged in the database to look at the provider or refer an injured worker to a better provider in states where such action is allowed. When medical providers follow treatment plans based on EBM, the methods and systems can refer patients to that provider.

The compliance scores can be incorporated into a predictive model as an input feature to improve accuracy on reserves and ensure appropriateness of case manager referrals. That is, a model can be developed that receives compliance scores as input and operates to flag records for case manager referrals. The case manager referrals can be directed to an appropriate case manager client device from the server, which can implement the model. The case manager client device can be associated with multiple levels of case managers, e.g., a nurse case manager, a senior nurse case manager, a doctor case manager, a senior doctor case manager, a fraud prevention case manager, an opioid review case manager, or the like.

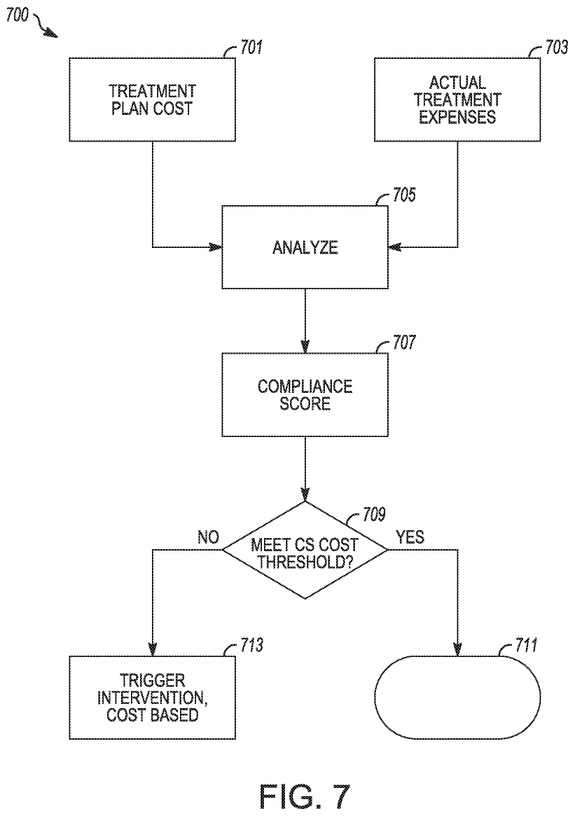

FIG. 7 is a flow diagram generally illustrated a method 700 for analyzing stored and derived information, which can be stored in the database, relating to EBM based treatments and actual treatments to produce a compliance score using costs. At operation 701, the ideal treatment plan cost is provided for a given diagnosis. In an example embodiment, the ideal treatment plan cost is the aggregate costs associated with treatment plan developed in method 500.

At operation 703, the actual treatment cost for an individual and an individual diagnosis is provided. The actual treatment plan cost is directed to an individual for a specific diagnosis.

At operation 705, the actual treatment cost is analyzed relative to the ideal treatment plan cost and outputs the compliance score to operation 707. The compliance score represents actual treatment costs divided by the ideal treatment cost. The resultant score can be output as a percentage of costs for a given injury (diagnosis). This cost-based compliance can have a higher sensitivity than other compliance scores. In the alternative, the analysis of the treatment costs 701, 703 can be performed on a basis of the medical provider costs to produce a provider cost compliance score. The analysis of the treatments 701, 703 can be performed on a basis of the medical clinic costs to produce a medical clinic compliance score.

At operation 709, the compliance cost score is compared to a compliance cost threshold. If the compliance cost score meets the threshold, then the process 700 ends at 711. If the compliance cost score does not meet the threshold, then an intervention is triggered at operation 713. The intervention can be the remedial actions at level 402 (FIG. 4).

In some embodiments, a machine learning system, such as a neural network executing a machine learning algorithm, may receive various inputs, such as the data record described herein. The machine learning system may be trained using the data records (e.g. large quantities of data comprising a plurality of data records capture over a relatively long period). The machine learning system may be configured (e.g., having been trained using the data records) to analyze the compliance score and identify claim risk associated with the claim. Additionally, or alternatively, the machine learning system may determine claim risk for high cost and extended duration at any time (e.g., as the claim matures).

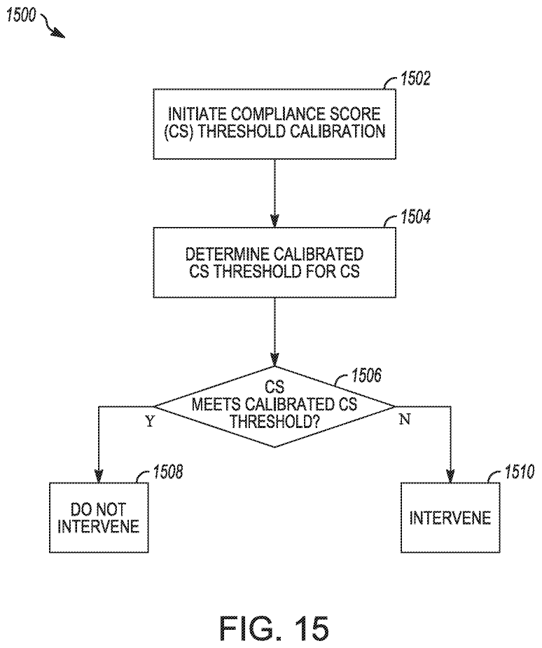

In some embodiments, if intervention is triggered at 613 and/or 713, a method 1500, as is generally illustrated in FIG. 15 may begin at 1502. The method 1500 may be configured to calibrate thresholds for the compliance score. At operation 1502, a compliance score threshold calibration is initiated. The compliance score threshold calibration may include initiating a machine learning algorithm configured to calibrate the compliance score threshold. The machine learning algorithm may be executed on any suitable computing device which may execute include any suitable machine learning system, neural network system, artificial intelligence system, and the like. The machine learning algorithm may be trained using a plurality of data entries corresponding to a plurality of claims. The machine learning algorithm may be trained using any suitable method or technique.

In some embodiments, the machine learning algorithm accesses a relatively large database. The database may include features similar to those of the database 204 and/or additional or fewer features. In some embodiments, the database may include significantly more data than the database 204 relating to a plurality of other aspects of the claims than are stored in the database 204. The database may comprise a number of tables for storing data and/or data may be stored in the database in other types of data structures (e.g., as an object-oriented database). The database may include a relatively large amount of data (e.g., millions or more data entries) stored in the tables. For example, the database may include an injured worker (e.g., patient) table, a treatment table, a provider table, a claim event table, and a remedial action table, as described with respect to the database 204.

The injured worker table may store data relating to the injured worker, e.g., identifying information, diagnosis, doctor, other medical providers, medical treatment, dates of injury and treatment, individual compliance score, which can be derived from the diagnosis and treatment being performed. Additionally, or alternatively, the injured worker table may store data relating to a geographic location of the injured worker, an age of the injured worker, a material status of the injured worker, other health characteristics of the injured worker, other suitable data relating to the injured worker, or a combination thereof.

The treatment table can include types of treatments that relate to a diagnosis, data corresponding to patient compliance of such treatments by geographic location, age, marital status, underlying health conditions, other suitable patient characteristics, or a combination thereof. The provider table can include identification information for an individual provider, their practice, medical specialty, historical treatments in total, specific treatments for individuals, patient compliance (e.g., by geographic location, age, marital status, underlying health conditions, other suitable patient characteristics, or a combination thereof for the provider), and billing information. The claim event table can store all events that relate to an individual or an injury to an individual. The remedial action table can store all remedial actions taken for a claim and the results therefrom.

The database can also store data relating to the compliance score, e.g., a compliance score table and a compliance score threshold table. The compliance score table can store the compliance score on a per injury basis, e.g., a compliance score value is assigned to any triggering claim event, i.e., an injury to a worker. The compliance score value is a compliance score derived from the analysis of best practices. The best practices are derived from historical outcomes and medical research and literature. The compliance score threshold table stores the threshold at which an individual's compliance score will trigger remedial action. An individual's compliance score is calculated based on the diagnosis and treatments/care provided which can be stored in the patient (injured worker) table.

The database can also store data relating medical treatment and classification, e.g., the Evidence Based Medicine Reference (EBM) table, the Current Procedural Terminology (CPT) table, the International Classifications of Diseases (ICD) code table, the National Drug Code (NDC) table. The EBM table stores information to determine, at the time of treatment, the frequency and extent of services presumed to be medically necessary and appropriate for compensable injuries. The CPT table stores information to identify medical, surgical, and diagnostic services and is designed to communicate uniform information about medical services and procedures among physicians, coders, patients, accreditation organizations, and payers for administrative, financial, and analytical purposes.

The International Classifications of Diseases (ICD) code table stores information to identify diseases, disorders, symptoms, poisonings, adverse effects of drugs and chemicals, injuries and other reasons for patient encounters. Diagnostic coding in table is the translation of written descriptions of diseases, illnesses and injuries into codes from a particular classification. In medical classification, diagnosis codes are used as part of the clinical coding process alongside intervention codes. Both diagnosis and intervention codes are assigned by a health professional trained in medical classification such as a clinical coder or Health Information Manager. The database may include additional tables storing data in addition to those described herein and may store data for thousands or millions of patients, injuries, treatments, results, compliance, and the like.

At operation 1504, the machine learning algorithm analyzes the data stored in the database and determines a specific threshold for the compliance score corresponding to the injured worker. The machine learning algorithm provides a specific compliance score threshold for the specific injured worker based on a plurality of data entries corresponding to the specific injured worker and a plurality of data entries corresponding to other patients. For example, the machine learning algorithm may analyze treatments provided providers in a similar geographic region to the injured worker, treatments provided to other patient in a similar geographic region and or a similar age, treatments provided to other patients of a similar socioeconomic status, other suitable data, or a combination thereof. The machine learning algorithm outputs a specific compliance score threshold based on the analysis of the data and the training of the machine learning algorithm. The compliance score threshold is calibrated to the specific compliance score threshold.

At operation 1506, the compliance score is compared to the calibrated compliance score threshold. If the compliance cost score meets the calibrated compliance score threshold, then the method 1500 does not intervene at operation 1508. If the compliance cost score does not meet the threshold, then the method 1500, at operation 1510, continues the intervention triggered in operation 613 and/or operation 713.

Software Architecture

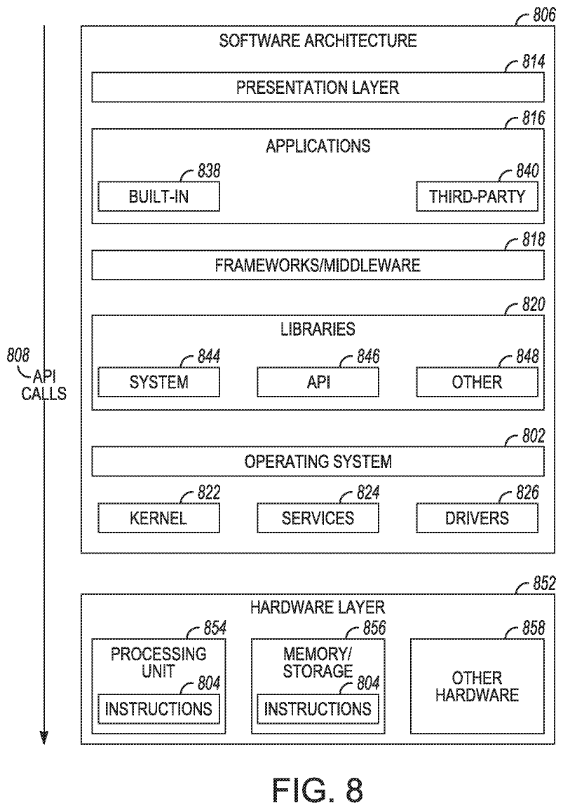

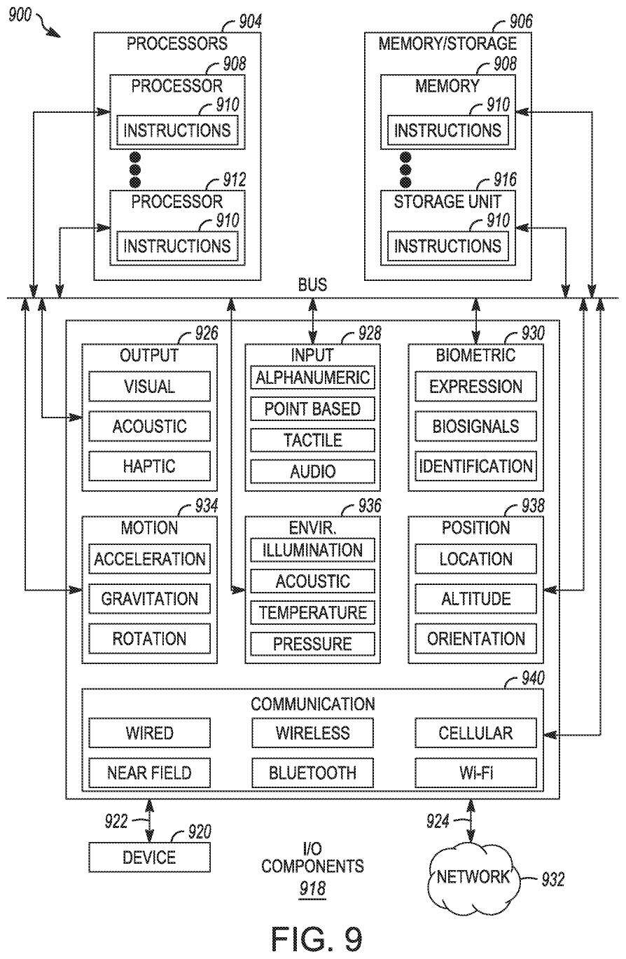

FIG. 8 generally illustrates a block diagram illustrating a software architecture 806, which may be used in conjunction with various hardware architectures herein described. FIG. 8 is a non-limiting example of a software architecture and it will be appreciated that many other architectures may be implemented to facilitate the functionality described herein. The software architecture 806 may execute on hardware such as machine 900 of FIG. 9 that includes, among other things, processors 904, memory 914, and I/O components 918. A representative hardware layer 852 is illustrated and can represent, for example, the machine 900 of FIG. 9. The representative hardware layer 852 includes a processing unit 854 having associated executable instructions 804. Executable instructions 804 represent the executable instructions of the software architecture 806, including implementation of the methods, components and so forth described herein. The hardware layer 852 also includes memory or storage modules memory/storage 856, which also have executable instructions 804. The hardware layer 852 may also comprise other hardware 858. The executable instructions 804 can include operation tasks to retrieve and develop ideal treatment plans from evidence-based medicine sources. Each treatment plan may be individualized for a single injury or a single illness. The instructions can develop the ideal treatment plan from historical data in a medical care database, including treatments received and outcomes. The instructions can further analyze the actual treatment received by patient in view of compliance or divergence from the ideal treatment plan. The instructions can flag an actual treatment or course of treatment and all data fields associated with the treatment that does not meet a threshold value. The instructions can trigger an intervention action, e.g., alerting through a communication network from the hardware layer to a client device. The flag can trigger visual indicators on a graphical user interface when the healthcare data is shown on a remote display. The instructions 804 can also operate to track costs associated with treatments and provide a compliance score related to costs. Data records can be flagged when the costs do not match an ideal cost for an individual treatment plan.

As used herein, the term “component” may refer to a device, physical entity or logic having boundaries defined by function or subroutine calls, branch points, application program interfaces (APIs), or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions.

Components may constitute either software components (e.g., code embodied on a machine-readable medium) or hardware components. A “hardware component” is a tangible unit capable of performing certain operations and may be configured or arranged in a certain physical manner to produce the compliance score and to output graphical user interfaces using the compliance score. In some embodiments, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein. A hardware component may also be implemented mechanically, electronically, or any suitable combination thereof. For example, a hardware component may include dedicated circuitry or logic that is permanently configured to perform certain operations, e.g., to gather EBM, to gather treatment information, compute a compliance score, and output graphical user interfaces.

A hardware component may be a special-purpose processor, such as a Field-Programmable Gate Array (FPGA) or an Application Specific Integrated Circuit (ASIC). A hardware component may also include programmable logic or circuitry that is temporarily configured by software to perform certain operations. For example, a hardware component may include software executed by a processor or other programmable processor. Once configured by such software, hardware components become specific machines (or specific components of a machine) uniquely tailored to perform the configured functions and are no longer processors. It will be appreciated that the decision to implement a hardware component mechanically, in dedicated and permanently configured circuitry, or in temporarily configured circuitry (e.g., configured by software) may be driven by cost and time considerations.

A processor may be, or include, any circuit or virtual circuit (a physical circuit emulated by logic executing on an actual processor) that manipulates data values according to control signals (e.g., “commands”, “op codes”, “machine code”, etc.) and which produces corresponding output signals that are applied to operate a machine. A processor may, for example, be a Central Processing Unit (CPU), a Reduced Instruction Set Computing (RISC) processor, a Complex Instruction Set Computing (CISC) processor, a Graphics Processing Unit (GPU), a Digital Signal Processor (DSP), an Application Specific Integrated Circuit (ASIC), a Radio-Frequency Integrated Circuit (RFIC) or any combination thereof. A processor may further be a multi-core processor having two or more independent processors (sometimes referred to as “cores”) that may execute instructions contemporaneously. The processor as used herein may be a hardware component, which is in at least one of the devices, systems, servers and the like. The processor may include multiple cores and may be spread across multiple devices. The processor includes circuitry to execute instructions relating to the methods and structures described herein for determining relationships and outputting relationship data that is used by various devices and their users.

Accordingly, the phrase “hardware component” (or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein, e.g., related to compliance scoring. For example, where a hardware component comprises a processor configured by software to become a special-purpose processor, the processor may be configured as respectively different special-purpose processors (e.g., comprising different hardware components) at different times. Software accordingly configures a particular processor or processors, for example, to constitute a particular hardware component at one instance of time and to constitute a different hardware component at a different instance of time. Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In embodiments in which multiple hardware components are configured, or instantiated, at different times, communications between such hardware components may be achieved, for example, through the storage and retrieval of information in memory structures to which the multiple hardware components have access.

For example, one hardware component may perform an operation and store the output of that operation in a memory device to which it is communicatively coupled. A further hardware component may then, at a later time, access the memory device to retrieve and process the stored output. Hardware components may also initiate communications with input or output devices, and can operate on a resource (e.g., a collection of information). The various operations of example methods described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions described herein. As used herein, “processor-implemented component” refers to a hardware component implemented using one or more processors. Similarly, the methods described herein may be at least partially processor-implemented, with a particular processor or processors being an example of hardware. For example, at least some of the operations of a method may be performed by one or more processors or processor-implemented components.

Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an Application Program Interface (API)). The performance of certain of the operations may be distributed among the processors, not only residing within a single machine, but deployed across a number of machines. In some embodiments, the processors or processor-implemented components may be located in a single geographic location (e.g., within a home environment, an office environment, or a server farm). In other embodiments, the processors or processor-implemented components may be distributed across a number of geographic locations.

In the architecture of FIG. 9, the software architecture 906 may be conceptualized as a stack of layers where each layer provides particular functionality. For example, the software architecture 906 may include layers such as an operating system 902, libraries 920, applications 916 and a presentation layer 914. Operationally, the applications 916 or other components within the layers may invoke application programming interface (API) calls 908 through the software stack and receive messages 912 in response to the API calls 908. The layers illustrated are representative in nature and not all software architectures have all layers. For example, some mobile or special purpose operating systems may not provide a frameworks/middleware 918, while others may provide such a layer. Other software architectures may include additional or different layers.

The operating system 902 may manage hardware resources and provide common services. The operating system 902 may include, for example, a kernel 922, services 924 and drivers 926. The kernel 922 may act as an abstraction layer between the hardware and the other software layers. For example, the kernel 922 may be responsible for memory management, processor management (e.g., scheduling), component management, networking, security settings, and so on. The services 924 may provide other common services for the other software layers. The drivers 926 are responsible for controlling or interfacing with the underlying hardware. For instance, the drivers 926 include display drivers, camera drivers, Bluetooth® drivers, flash memory drivers, serial communication drivers (e.g., Universal Serial Bus (USB) drivers), Wi-Fi® drivers, audio drivers, power management drivers, and so forth depending on the hardware configuration.

The libraries 920 provide a common infrastructure that is used by the applications 916 or other components or layers. The libraries 920 provide functionality that allows other software components to perform tasks in an easier fashion than to interface directly with the underlying operating system 902 functionality (e.g., kernel 922, services 924 or drivers 926). The libraries 920 may include system libraries 944 (e.g., C standard library) that may provide functions such as memory allocation functions, string manipulation functions, mathematical functions, and the like. In addition, the libraries 920 may include API libraries 946 such as media libraries (e.g., libraries to support presentation and manipulation of various media format such as MPREG4, H.264, MP3, AAC, AMR, JPG, PNG), graphics libraries (e.g., an OpenGL framework that may be used to render 2D and 3D in a graphic content on a display), database libraries (e.g., SQLite that may provide various relational database functions), web libraries (e.g., WebKit that may provide web browsing functionality), and the like. The libraries 920 may also include a wide variety of other libraries 948 to provide many other APIs to the applications 916 and other software components/modules.

The frameworks/middleware 918 (also sometimes referred to as middleware) provide a higher-level common infrastructure that may be used by the applications 916 or other software components/modules. For example, the frameworks/middleware 918 may provide various graphic user interface (GUI) functions, high-level resource management, high-level location services, and so forth. The frameworks/middleware 918 may provide a broad spectrum of other APIs that may be utilized by the applications 916 or other software components/modules, some of which may be specific to a particular operating system 902 or platform.

The applications 916 include built-in applications 938 or third-party applications 940. The third-party applications 940 may invoke the API calls 908 provided by the operating system 902 to facilitate functionality described herein.

The applications 916 may use built in operating system functions (e.g., kernel 922, services 924 or drivers 926), libraries 920, and frameworks/middleware 918 to create user interfaces to interact with users of the system. Alternatively, or additionally, in some systems interactions with a user may occur through a presentation layer, such as presentation layer 914. In these systems, the application/component “logic” can be separated from the aspects of the application/component that interact with a user.

FIG. 9 generally illustrates a block diagram illustrating components (also referred to herein as “modules”) of a machine 900, according to the principles of the present disclosure, able to read instructions from a machine-readable medium (e.g., a machine-readable storage medium) and perform any one or more of the methodologies discussed herein. Specifically, FIG. 9 shows a diagrammatic representation of the machine 900 in the example form of a computer system, within which instructions 910 (e.g., software, a program, an application, an applet, an app, or other executable code) for causing the machine 900 to perform any one or more of the methodologies discussed herein may be executed. As such, the instructions 910 may be used to implement modules or components described herein. The instructions 910 transform the non-programmed machine 900 into a particular machine 900 programmed to carry out the described and illustrated functions in the manner described. In alternative embodiments, the machine 900 operates as a standalone device or may be coupled (e.g., networked) to other machines. In a networked deployment, the machine 900 may operate in the capacity of a server machine or a client machine in a server-client network environment, or as a peer machine in a peer-to-peer (or distributed) network environment. The machine 900 may comprise, but not be limited to, a server computer, a client computer, a personal computer (PC), a laptop computer, a network router, a network switch, a network bridge, or any machine capable of executing the instructions 910, sequentially or otherwise, that specify actions to be taken by machine 900. Further, while only a single machine 900 is illustrated, the term “machine” shall also be taken to include a collection of machines that individually or jointly execute the instructions 910 to perform any one or more of the methodologies discussed herein.

The machine 900 may include processors 904, memory memory/storage 906, and I/O components 918, which may be configured to communicate with each other such as via a bus 902. The memory/storage 906 may include a memory 914, such as a main memory, or other memory storage, and a storage unit 916, both accessible to the processors 904 such as via the bus 902. The storage unit 916 and memory 914 store the instructions 910 embodying any one or more of the methodologies or functions described herein. The instructions 910 may also reside, completely or partially, within the memory 914, within the storage unit 916, within at least one of the processors 904 (e.g., within the processor's cache memory), or any suitable combination thereof, during execution thereof by the machine 900. Accordingly, the memory 914, the storage unit 916, and the memory of processors 904 are examples of machine-readable media.

As used herein, the term “machine-readable medium,” “computer-readable medium,” or the like may refer to any component, device or other tangible media able to store instructions and data temporarily or permanently. Examples of such media may include, but is not limited to, random-access memory (RAM), read-only memory (ROM), buffer memory, flash memory, optical media, magnetic media, cache memory, other types of storage (e.g., Erasable Programmable Read-Only Memory (EEPROM)) or any suitable combination thereof. The term “machine-readable medium” should be taken to include a single medium or multiple media (e.g., a centralized or distributed database, or associated caches and servers) able to store instructions. The term “machine-readable medium” may also be taken to include any medium, or combination of multiple media, that is capable of storing instructions (e.g., code) for execution by a machine, such that the instructions, when executed by one or more processors of the machine, cause the machine to perform any one or more of the methodologies described herein. Accordingly, a “machine-readable medium” may refer to a single storage apparatus or device, as well as “cloud-based” storage systems or storage networks that include multiple storage apparatus or devices. The term “machine-readable medium” excludes signals per se.

The I/O components 918 may include a wide variety of components to provide a user interface for receiving input, providing output, producing output, transmitting information, exchanging information, capturing measurements, and so on. The specific I/O components 918 that are included in the user interface of a particular machine 900 will depend on the type of machine. It will be appreciated that the I/O components 918 may include many other components that are not shown in FIG. 9. The I/O components 918 are grouped according to functionality merely for simplifying the following discussion and the grouping is in no way limiting. In some embodiments, the I/O components 918 may include output components 926 and input components 928. The output components 926 may include visual components (e.g., a display such as a plasma display panel (PDP), a light emitting diode (LED) display, a liquid crystal display (LCD), a projector, or a cathode ray tube (CRT)), acoustic components (e.g., speakers), other signal generators, and so forth. The input components 928 may include alphanumeric input components (e.g., a keyboard, a touch screen configured to receive alphanumeric input, a photo-optical keyboard, or other alphanumeric input components), point based input components (e.g., a mouse, a touchpad, a trackball, a joystick, a motion sensor, or other pointing instrument), tactile input components (e.g., a physical button, a touch screen that provides location or force of touches or touch gestures, or other tactile input components), audio input components (e.g., a microphone), and the like. The input components 928 may also include one or more image-capturing devices, such as a digital camera for generating digital images or video.

In some embodiments, the I/O components 918 may include biometric components 930, motion components 934, environmental environment components 936, or position components 938, as well as a wide array of other components. One or more of such components (or portions thereof) may collectively be referred to herein as a “sensor component” or “sensor” for collecting various data related to the machine 900, the environment of the machine 900, a user of the machine 900, or a combinations thereof.

Communication may be implemented using a wide variety of technologies. The I/O components 918 may include communication components 940 operable to couple the machine 900 to a network 932 or devices 920 via coupling 922 and coupling 924 respectively. For example, the communication components 940 may include a network interface component or other suitable device to interface with the network 932. In further examples, communication components 940 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 920 may be another machine or any of a wide variety of peripheral devices (e.g., a peripheral device coupled via a Universal Serial Bus (USB)). Moreover, the communication components 940 may detect identifiers or include components operable to detect identifiers.

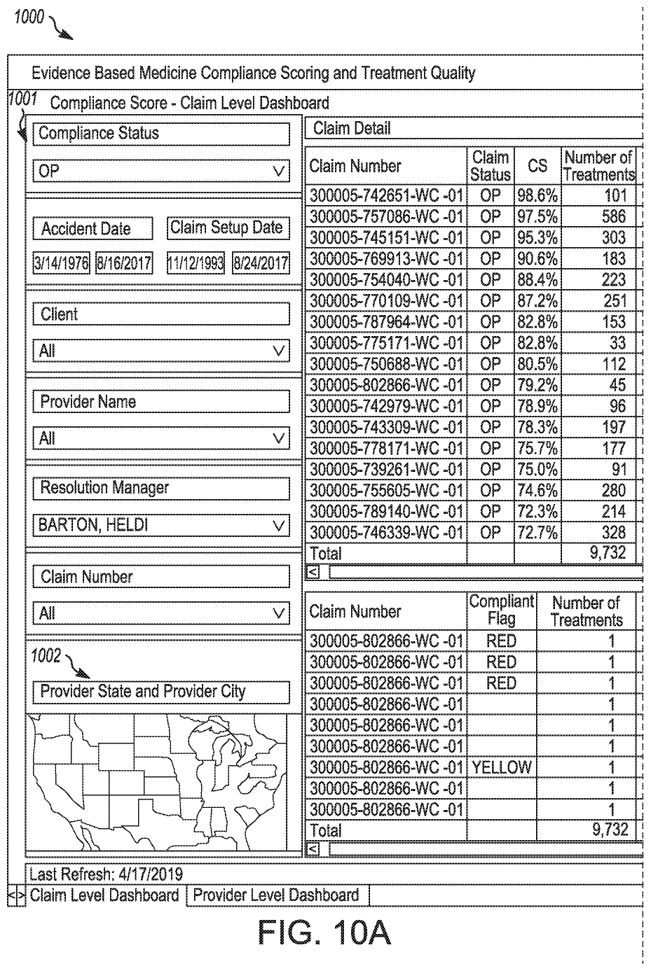

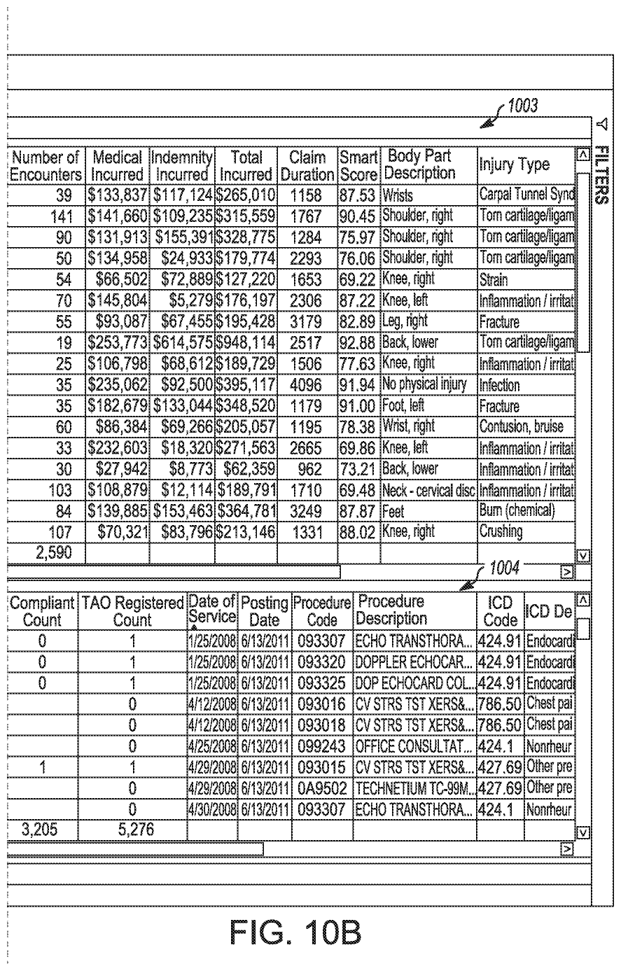

FIGS. 10A and 10B generally illustrate a schematic view of a graphical user interface 1000 on a display screen that uses the compliance score. The compliance score can be produced according to the teachings herein. The GUI 1000 shows various input boxes and data tables related to evidence based medicine compliance scoring and treatment quality as it relates to compliance scores at a claim level. The GUI 1000 includes a first frame 1001 that shows claim status, accident date, claim set up date, client, provider name, resolution manager and claim number, which are all enterable or selectable inputs to filter the data that is shown in the second frame 1002, third frame 1003, and the fourth frame 1004. The second frame 1002 shows the locations of the claims data overlaid on a map of the United States. It will be recognized that another country or region could be the subject of the map in frame 1002. The highlighted states indicate those in which there are claims being shown in the display. This provides a visualization that quickly conveys the states that include claims that are being displayed. The highlighted states include Washington and California in the west and Pennsylvania, Virginia, and North Carolina in the east. Upon selection of different data in any of frames, 1001, 1003, or 1004, the map would change an only highlight those geographic areas (here, states) in which a displayed claim is located.

Frame 1003 shows claim details for the individual claims, which represent a single injury or illness but may include multiple events during treatment. Frame 1003 shows a claim number, claim status, compliance score (which can be calculated as described herein), number of treatment, number of encounters, medical costs incurred, indemnity incurred, claim duration, smart score, body part description, and type of injury In the present example, the first compliance score threshold is set at a first threshold, here, 90%. The first four claims have a compliance score (CS) of greater than the first threshold and are notified using a first color indicator. In the present example, the second compliance score threshold is set at a second threshold value, here, 75%. The next ten claims have a compliance score (CS) of greater than the second threshold (75%) and less than the first threshold (90%). These are noted with a second visual indicator, e.g., a second color indicator, which is different from the first indicator. The remaining claims are shown as falling below the second threshold value. This third grouping of claims are noted with a third visual indicator, e.g., a third color indicator, which is different from the first indicator and the second indicator. The visual indicators can be green for those claims above the first threshold, yellow for the claims between the first threshold value and the second threshold value, and red for those below the second threshold value. There can be a fourth indicator, e.g., black, for those compliance scores that are amongst the lowest compliance scores. The indicators on the GUI provide a quick visual reference to the compliance scores that require attention, e.g., remedial actions 402 or decision support actions 402 in FIG. 4.

Frame 1004 shows data relating to a treatment detail along with the compliant flag. The compliant flag is based on the compliant score but simplifies to the flag value that corresponds to the indicator, e.g., color, red, yellow and green, in frame 1003. The treatment detail in frame 1004 can also show the claim number, the number of treatments, the compliant count, the TAO registered count, the date of service, the posting date, the procedure code, the procedure description, the ICD code and the ICD description.

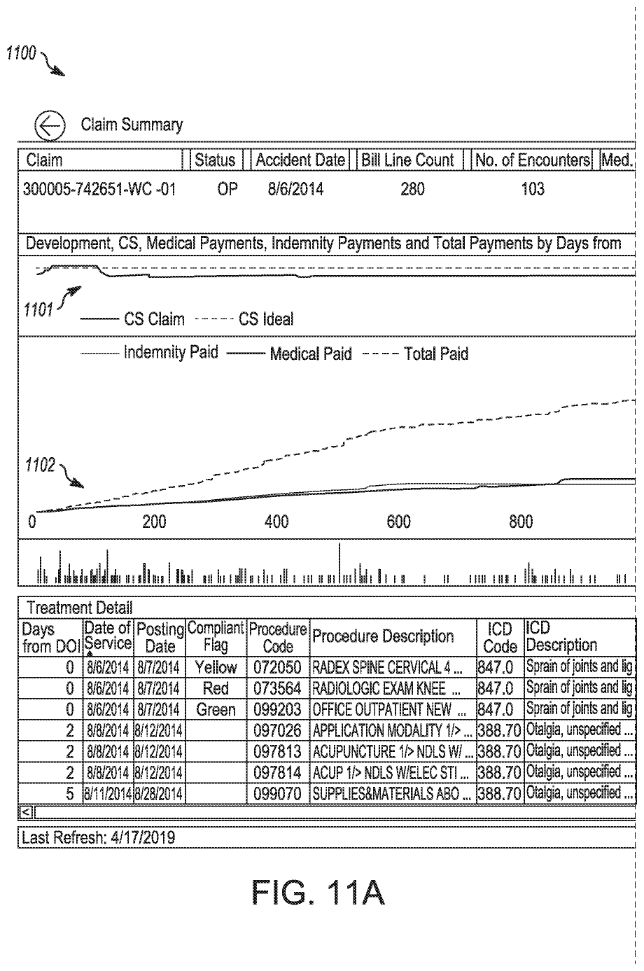

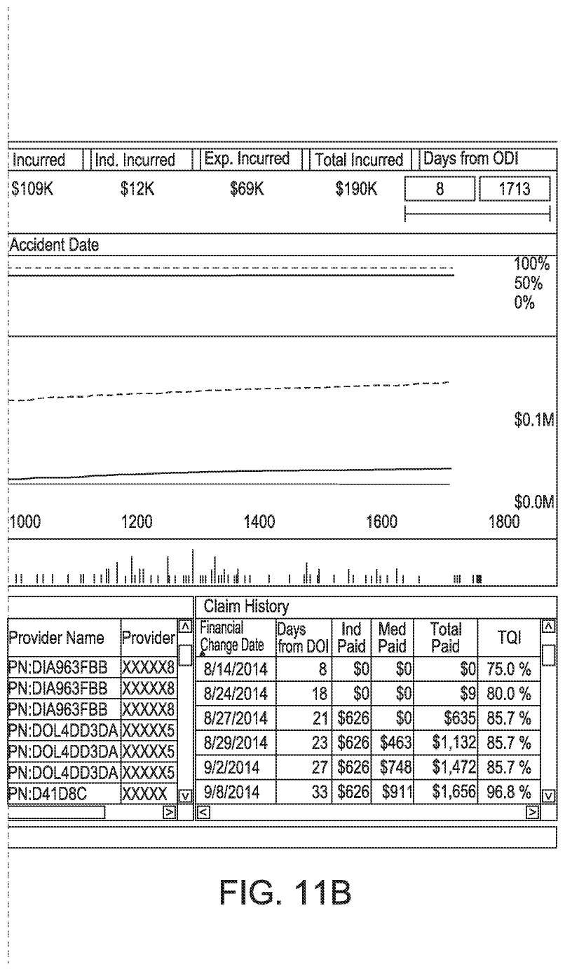

FIGS. 11A and 11B generally illustrate a schematic view of a graphical user interface 1100 on a display screen that uses the compliance score with relation to a single claim. A single claim, here claim no. 3000005-755605-WC-1, is shown over time. Various statistics associated with this claim are also shown, status, accident date, bill line count, number of encounters, medical costs incurred, indemnity costs, expenses incurred and the total cost incurred. Two graphs 1101, 1102 are shown on GUI 1100. Time is shown on the abscissa and is measured in days since the injury or illness for both graphs. The top ordinate of top graph 1101 is the actual compliance score with both the actual compliance score (CS claim) and the compliance threshold (CS ideal) shown on the graph. Here, the claim was in compliance for a short time, e.g., day 10 to about day 120, and then dropped below the compliance threshold for the reminder of the time. The second ordinate for the second graph 1102 represents the costs associated with the claim, with a line for the indemnity paid, medical paid and the running total paid.

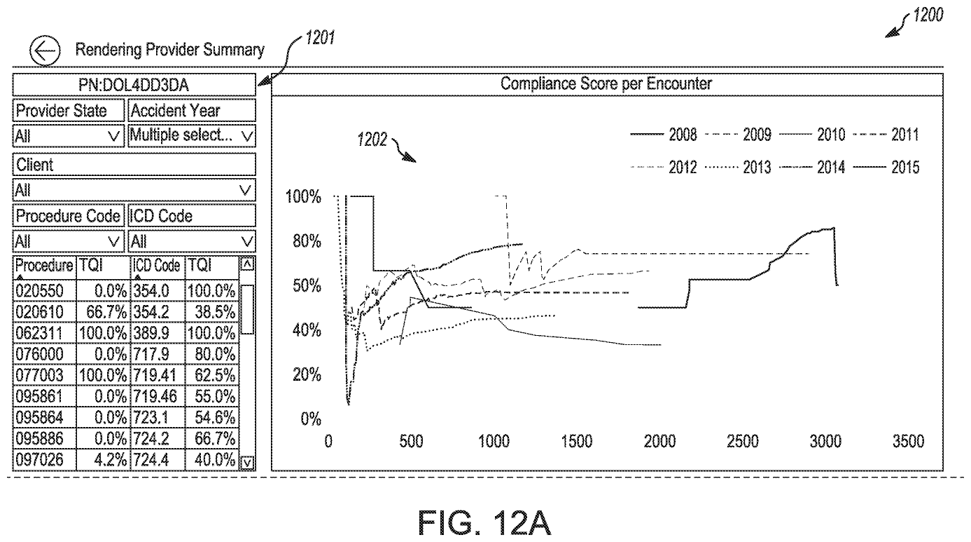

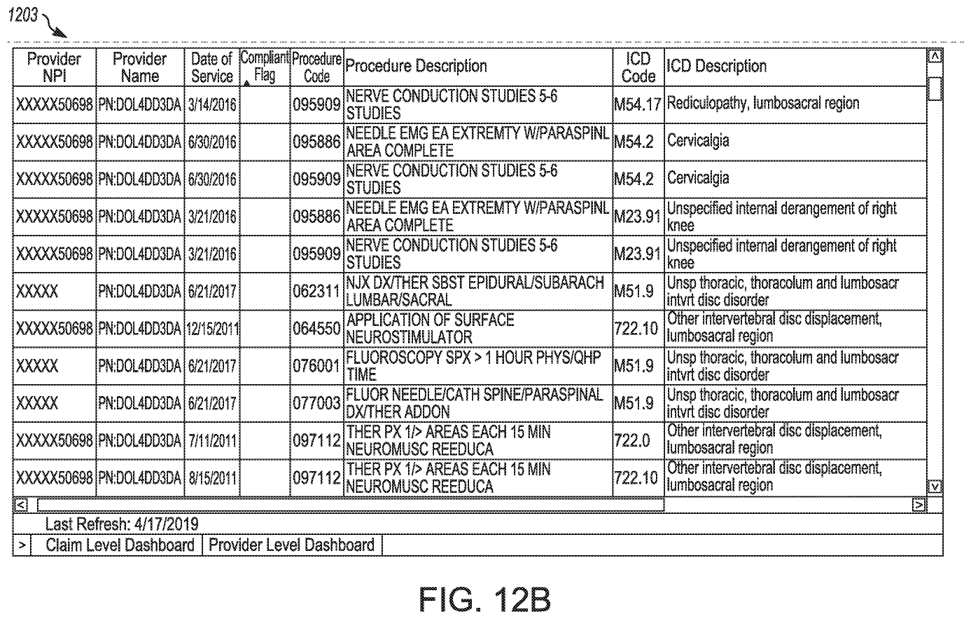

FIGS. 12A and 12B generally illustrate a schematic view of a graphical user interface 1200 on a display screen that uses the compliance score with relation to a provider. A first frame 1202 includes information related to a single medical provider, here provider number DOL4DFD3DA. The first frame 1202 includes the provider state field, the accident year (to be selected for display) field, a client field, a procedure code field and an ICD field. Each of the fields includes selectable data to limit that filters the information related to the provider and which can be displayed in the second and third frames 1202, 1203. The first frame also shows indicated values of the compliance score in the procedure code list and the ICD code list. The second frame 1202 shows a graph of the compliance score per encounter for each of the years that the provider provided a treatment event recorded in the database. The number of encounters is shown on the abscissa. The compliance score is shown in the ordinate. The third frame 1203 shows the details of the encounters (e.g., treatments) performed by the provider.

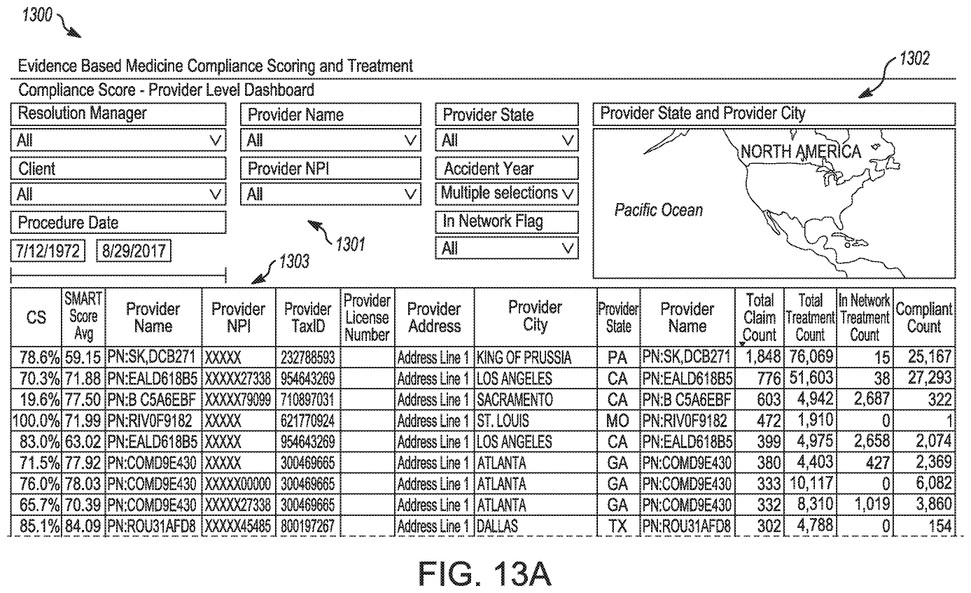

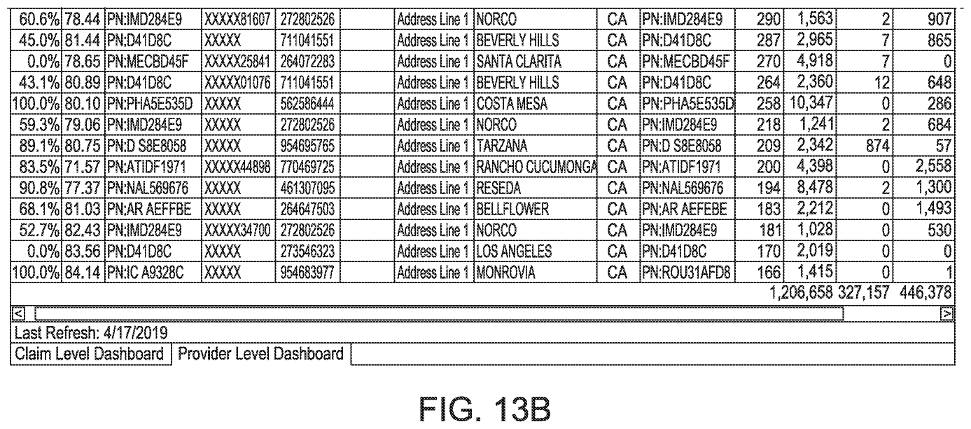

FIGS. 13A and 13B generally illustrate a schematic view of a graphical user interface 1300 on a display screen that uses the compliance score with evidence-based medicine on a provider level. The first frame defines filters for resolution manager, provider name, provider state, client, provider NPI, accident year, procedure date and in network flag. This are all defaulted to all of maximum range as shown. The second frame 1302 is a map. As the filters are at their default maximum values there is nothing shown on the map. The frame 1303 lists all of the encounters, e.g., treatments, along with their compliance scores with indicators.

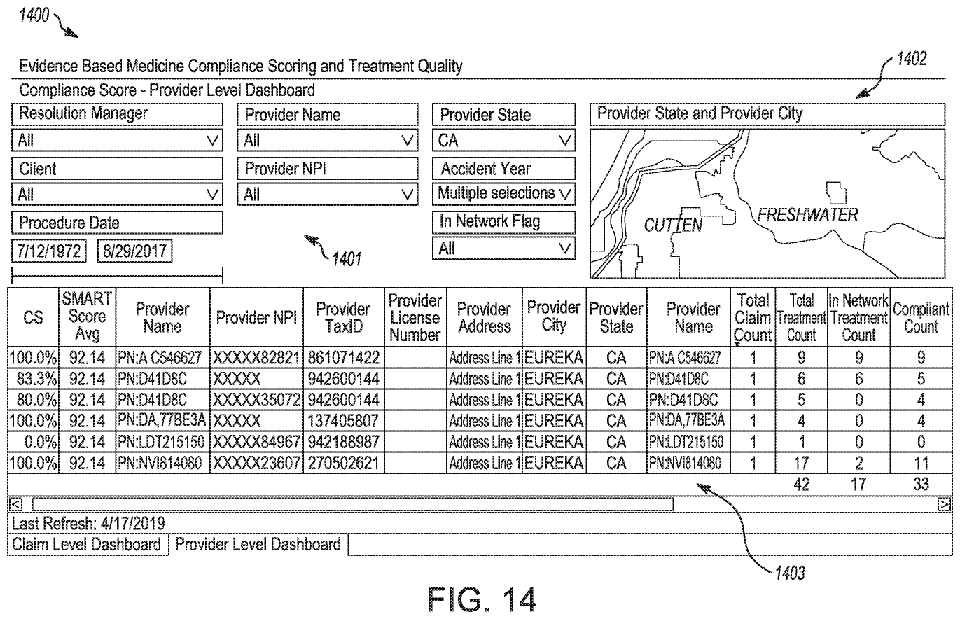

FIG. 14 generally illustrates a schematic view of a graphical user interface 1400 on a display screen that uses the compliance score with evidence based medicine on a provider level, similar to GUI 1300 but with a filter added and the map controlled to limit the number of results. Frame 1401 differs from frame 1301 as the provider state is set to California (CA). This limits the provider details in the third frame 1403 to the encounters in California, i.e., a first geographic region. The second frame 1402 is also changed from second frame 1302 and is zoomed into a specific geographic region of California. This applies a second filter to the encounters and only list the encounters in that region. Thus, the results can be filtered in real time while showing the resultant filtered data, which can be done on a geographic basis. A person who is in need of medical care in a specific region can be advised to go to the providers who have better outcomes as measured by the compliance score (CS). In this limited geographic region, there are three providers with perfect compliance scores. A person should be encouraged to use these providers for best outcomes. There is one provider with a zero compliance score. A person should be discouraged from using this provider, as there are five providers with better compliance scores for a better outcome.

The graphical user interfaces 1000, 1100, 1200, 1300, and 1400 can be generated using the hardware described herein, e.g., the server 110, processing unit 854, processors 904 or the like.

The present disclosure relates the compliance score to claims, e.g., injury of illness claims. A claim can be the initial notification to an interested party, e.g., an insurer, an employer, a governmental agency, a health care organization, that an injury or illness occurred and may be covered under insurance or a program. The claims process for an on the job injury or illness can be as follows. Immediately upon injury, the worker obtains the necessary medical treatment from a HCW (e.g., provider) and notifies his/her supervisor about the accident and how it occurred. The worker notifies the employer of the accident in writing, as soon as possible, but within a mandated time period, e.g., two weeks, 30 days, or 60 days.

The worker typically files a claim with the appropriate government agency. This filing must be done within a time period, e.g., six months, one year, two years from the accident, or within two years after the worker knew or should have known, that the injury was related to employment. Within a short time period from the accident, e.g., 48 hours, 72 hours or one week, the HCW completes a preliminary medical report, which may be filed with the government agency. Within a time period after the accident, e.g., multiple days, the employer reports the injury to the government agency and the insurance company. Within further time period after this report, the insurer provides the injured worker with a written statement of his/her rights under the law. In addition, if the insurer requires claimants to use a network it has contracted with to obtain diagnostic tests or treatment, the insurer notifies the claimant of the name and contact information for the network at the same time it sends the written statement of his/her rights or immediately if that time has passed.

The insurer can use compliance scores to select the network of providers to the worker. If the injury or illness exceeds a time period, the insurer begins the payment of benefits. If the claim is being disputed, the insurer must inform the worker (claimant) and the appropriate governmental agency. In an example, the insurer continues to make payments of benefits to the injured employee (if the case is not being disputed), every 2 weeks. The insurer must notify the governmental agency when compensation is stopped or modified. The insurer receives medical reports as treatment progresses. All of this information may be stored in the database of the insurer and gathered using devices and APIs.

As described herein, the compliance scoring process generates a unique series of statistically valid outputs that describe and rank order in actionable detail, a wide range of medical care scenarios commonly encountered in treating the types of injuries related to the administration of insurance claims (e.g., workers' compensation insurance claims). Compliance-score-derived metrics can be easily understood, standardized for benchmarking against a variety of external standards and measured at any time at the provider or claim level, or aggregated across contracted provider networks or claim populations.

Compliance score outputs can be used in a variety of ways. Selected views of Compliance score metrics are used in real time by insurance (workers' compensation) claim adjusters to identify open, active claims which are not performing within a desired compliance score range relative to a mismatch of treatment and the diagnosis and overall health condition of the claimant. The compliance score information is then deployed to guide an immediate intervention in the form of a peer review or other appropriate forensic review such as an Independent Medical Examination or a Functional Medical Examination. The use of compliance scores makes such claim situations significantly easier to identify and characterize early in the claim process. This can have serious cost, duration and outcome ramifications to the benefit of the claimant/injured worker and the payer. Simply stated, having a benchmark compliance score value for a specific injury and comparing that benchmark compliance score value against a claim specific compliance score allows the present system to flag a computer record associated with the specific claim for further investigation or review.

Used in the aggregate, compliance score metrics are a unique resource which can materially improve the maintenance of contracted provider networks by allowing the administrator to identify providers with superior compliance score for preferential contracting and active direction of injured workers, as permitted by local regulations. Without the compliance score process the identification of “gold standard” providers for preferential contracting is difficult and uncertain. In an example, the preferential contracting can be based on both geographic area and the compliance scores

The present inventors have identified that network administrators and claim managers who have been tasked with keeping up with the gamut of medical literature have been overwhelmed. Medical literature grows exponentially and can include new practice patterns, coding strategies, pharmaceuticals, and scientific research with over 5,000 medical journal articles published every month, 68,000 diagnoses codes (ICD-10), 8,000 procedural codes (CPT) and 1,500 FDA approved medications in the market (NDC).

Evidence-Based Medicine can be used in the present disclosure to develop compliance scoring. Several tools and references developed by physicians and healthcare professionals help caregivers and claims professionals make sense of this information. These standards have been codified and refined over decades by gathering, evaluating, and sharing battle-tested, proven clinical data. The process of developing medical practices based on the best available evidence from scientific research is known as evidence-based medicine (EBM). To date, network administrators and claim managers have to manually compare reams of claim data to available EBM guidelines. In workers' compensation the EBM guidelines are used as a yardstick to measuring the type, frequency, timing and duration of expected care. The intent of the EBM guidelines is to combine best practice protocols and optimal care pathways to avoid potentially harmful, inappropriate care for injured worker. They are best utilized by claims professionals to help them navigate the welter of highly variable local practices.

The workers' compensation Official Disability Guidelines, which are developed and published by MCG, may be used as a resource for the EBM component in the compliance score. The Official Disability Guidelines guide has been officially adopted by a large number of states and is generally recognized as authoritative in all states in evaluating work related disabilities.

The compliance score is a quantifiable measure value that involves mapping the EBM guidelines directly onto the medical billing data to derive a simple and easy to use compliance score for a given diagnosis, select provider or select clinic. The compliance score represents the percentage of care out of the total treatment plan for a given injury that complies with the (workers' compensation or other suitable insurance or treatment claim) treatment guidelines. It may be reported as a percentage from 1 to 100, or a normalized score from zero to one. Compliance score is applied at the claim or medical provider level and serves as a proxy for quality of care. At a basic level, compliance score is calculated by dividing the total cost or number of treatment compliant actions with the evidence based guidelines by the total cost or number of all treatment on the claim or associated with a provider.

The compliance score can be benchmarked as the raw compliance score has limited meaning by itself. The second part of the compliance score process involves benchmarking compliance score scores both nationally and regionally (to account for regional deviations in practice patterns) against a massive reference claims database containing millions of (workers' compensation) insurance claims. This statistical normalization process sorts the raw scores into quality-descriptive tranches appropriate to the state and the medical specialty represented, as well as the clinical complexity (or severity mix) of the claim (including claimant age and the presence of certain common co-morbidity factors).

The compliance score can be validated to prove its authenticity and potential impact, e.g., validation by peer-reviewed scientific research. It is believed that claims having higher percentage of medical treatments determined as “appropriate” vs. “inappropriate” by the Evidence Based Medicine Reference Guidelines (e.g., the Official Disability Guidelines and/or other suitable guidelines or standards) result in better outcomes, lower cost and less time out of work for the injured workers.

The compliance score can be deployed to open claims, e.g., claims for a patient still undergoing treatment. The compliance score intersects the management of workers' compensation claims via a dedicated decision support system which regularly reviews ongoing claims in near real time. The system reviews and scores the actual clinical services billed by the provider (CPT codes) against the diagnosis codes attached to the claims. Each review provides a full retrospective view of all clinical procedures to date. New compliance scores which exceed thresholds established (and revised) for the claim jurisdiction and claim severity index are referred to a group of specially trained registered nurses for a clinical review and possible referral to the managing adjuster for intervention.

The present systems and methods described herein can use the compliance scores to provide relative indication of the quality of care provided to the patient. The compliance score provides a care indicator that combines evidence based medicine with a claimant medical data, for example, a large data set containing millions of individual treatments and, in the case of workers' compensation, millions of on-the-job injuries. Compliance score can be an objective measure that is relative to an optimum treatment that provides quality outcomes.

The present systems and methods can be used to identify bad actors in the treatment, e.g., medical providers committing fraud or over prescribing pain medications such as opioids. The use of compliance scores can possibly reduce costs and get a worker back to their job on a timely basis while providing quality care. The compliance score can identify unnecessary treatments and inappropriate medications.

The present disclosure sets forth a fundamental change in the claim's management methods and systems. The present disclosure is designed to bring improved state-of-the-art, safe and effective care to medical insurance claims and moves away from the traditional 30-60-90-day reviews which rely on reactive intervention strategies driven by claim severity and treatment cost, and moves toward a more proactive value-based and clinical quality driven approach, using an objective compliance score to flag potential issues in records and on graphical user interfaces. The present disclosure derives a treatment schedule based on evidence-based medical investigation and science and compares the treatment schedule to an actual treatment to produce a compliance score. Compliance scores can be used to improve quality of care and ensure that it is optimal and consistent from clinician to clinician and place to place. The compliance score can trigger certain metrics and graphical indicators to identify those treatments, care providers, clinics or combinations thereof that meet a compliance threshold or do not meet a compliance threshold. It is recognized that medical care is too complicated to be shoved into a cookie cutter regime of rigid adherence. The compliance score is not a rigid calculation that something is wrong with treatment, only that in certain circumstances it might be wrong. Therefore, the compliance score is an indicator to flag a particular treatment schedule, provider or clinic for review. The flag can be in the data base or shown on a display.

Compliance score as used in some examples described herein measures how closely given historical treatments follow an ideal treatment plan. The historical treatments can be for an individual. The ideal treatment plan can be derived from historical medical data and evidence based medicine. The compliance score can be a measure of how close the actual historical treatments follow the ideal treatment plan. The compliance score can be a normalized score from a minimum to a maximum, e.g., zero to one. The compliance score can be matching expressed as a percentage with zero percent being no match and one hundred percent being an exact match. The compliance score can also be a measure of divergence from the ideal treatment, which can be expressed as an inverse of the matching examples. The compliance score can also be a quantitative measure of how many times the actual treatment varied from the ideal treatment. These are examples of how to measure compliance or divergence of the actual treatment from the ideal treatment. The present disclosure is not limited to the explicit examples of compliance scoring; other scoring systems are within the scope of the present disclosure.

In some embodiments, a system includes a processor and a memory component. The memory component having medical data records and instructions stored thereon, when executed by the processor, causes the processor to: compute a compliance score from an ideal treatment plan and an actual treatment, the actual treatment being stored in the medical data records; determine whether the compliance score is greater than a compliance threshold; in response to a determination that the compliance score is greater than the compliance threshold, store a pass flag with a data record of the actual treatment; and in response to a determination that the compliance score is less than the compliance threshold store a fail flag with the data record of the actual treatment and control a graphical user display to indicate the data record includes the fail flag.

In some embodiments, the instructions further cause the processor to, in response to storing a pass flag with the data record, control the graphical user display to indicate the data record includes the pass flag. In some embodiments, the instructions further cause the processor to compute costs for the ideal treatment plan based on a geographic area and costs for the actual treatment received for a same injury to an individual. In some embodiments, the medical data records include insurance claim records for an individual. In some embodiments, the instructions further cause the processor to control the graphical user display by limiting the graphical user display based on geographic area. In some embodiments, the instructions further cause the processor to trigger remedial events when the fail flag is generated or present in the data record. In some embodiments, the instructions further cause the processor to trigger decision support events when the fail flag is generated or present in the data record.

In some embodiments, a method includes receiving medical data records and computing a compliance score from an ideal treatment plan and an actual treatment, the actual treatment stored in medical data records. The method also includes determining whether the compliance score is greater than a compliance threshold and, in response to a determination that the compliance score is greater than the compliance threshold, storing a pass flag with a data record of the actual treatment. The method also includes, in response to a determination that the compliance score is less than the compliance threshold storing a fail flag with the data record of the actual treatment and controlling a graphical user display to indicate the data record includes the fail flag.

In some embodiments, the method also includes, in response to storing a pass flag with the data record, controlling the graphical user display to indicate the data record includes the pass flag. In some embodiments, the method also includes computing costs for the ideal treatment plan based on a geographic area and costs for the actual treatment received for a same injury to an individual. In some embodiments, the medical data records include insurance claim records for an individual. In some embodiments, the method also includes controlling the graphical user display by limiting the graphical user display based on geographic area. In some embodiments, the method also includes triggering remedial events when the fail flag is generated or present in the data record. In some embodiments, the method also includes triggering decision support events when the fail flag is generated or present in the data record.

In some embodiments, a system includes a processor and a memory component. The memory component having medical data records and instructions stored thereon, when executed by the processor, causes the processor to: receive an ideal treatment plan corresponding to an injury of an individual; identify an actual treatment in the medical data records corresponding to the ideal treatment plan; generate, using a machine learning system, a compliance score based on the ideal treatment plan and the actual treatment; determine whether the compliance score is greater than a compliance threshold; in response to a determination that the compliance score is greater than the compliance threshold store a pass flag with a data record of the actual treatment and control a graphical user display to indicate the data record includes the pass flag; and in response to a determination that the compliance score is less than the compliance threshold store a fail flag with the data record of the actual treatment and control the graphical user display to indicate the data record includes the fail flag.

In some embodiments, the instructions further cause the processor to compute costs for the ideal treatment plan based on a geographic area and costs for the actual treatment received for a same injury to the individual. In some embodiments, the medical data records include insurance claim records for the individual. In some embodiments, the instructions further cause the processor to control the graphical user display by limiting the graphical user display based on geographic area. In some embodiments, the instructions further cause the processor to trigger remedial events when the fail flag is generated or present in the data record. In some embodiments, the instructions further cause the processor to trigger decision support events when the fail flag is generated or present in the data record.

In some embodiments, a system includes a processor and a memory component. The memory component includes medical data records and instructions stored thereon that, when executed by the processor, causes the processor to: receive an ideal treatment plan corresponding to an injury of an individual; identify an actual treatment in the medical data records corresponding to the ideal treatment plan; generate, a compliance score based on the ideal treatment plan and the actual treatment; determine whether the compliance score is greater than a compliance threshold; in response to a determination that the compliance score is greater than the compliance threshold: store a pass flag with a data record of the actual treatment; and control a graphical user display to indicate the data record includes the pass flag; in response to a determination that the compliance score is less than the compliance threshold: store a fail flag with the data record of the actual treatment; control the graphical user display to indicate the data record includes the fail flag; and trigger intervention in response to the fail flag being stored in the data record of the actual treatment; and in response to intervention being triggered: generate, using machine learning, a calibrated compliance threshold corresponding to the compliance score; in response to a determination that the compliance score is less than the calibrated compliance threshold, continue the triggered intervention.

In some embodiments, the instructions further cause the processor to compute costs for the ideal treatment plan based on a geographic area and costs for the actual treatment received for a same injury to the individual. In some embodiments, the medical data records include worker's compensation records for the individual. In some embodiments, the instructions further cause the processor to in response to a determination that the compliance score is greater than the calibrated compliance threshold, discontinue the triggered intervention. In some embodiments, the instructions further cause the processor to trigger remedial events in response to intervention being triggered. In some embodiments, the instructions further cause the processor to trigger decision support events in response to intervention being triggered.

In some embodiments, The compliance score (CS) process generates a unique series of statistically valid outputs which describe and rank order, in actionable detail, a wide range of medical care scenarios commonly encountered in treating the types of injuries related to the administration of insurance claims (e.g., workers' compensation insurance claims). CS-derived metrics can be easily understood, standardized for benchmarking against a variety of external standards and measured at any time at the provider or claim level or aggregated across contracted provider networks or claim populations.

It should be understood that one or more processors may perform the systems and methods described herein. The one or more processors may include any suitable processor or processors, such as those described herein. The one or more processors may be in communication with a memory. The memory may include instructions that, when executed by the one or more processors, cause the one or more processors to perform the systems and methods described herein. The memory may comprise a single disk or a plurality of disks (e.g., hard drives), and includes a storage management module that manages one or more partitions within the memory. In some embodiments, memory may include flash memory, semiconductor (solid state) memory or the like. The memory may include Random Access Memory (RAM), a Read-Only Memory (ROM), or a combination thereof.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

The word “example” is used herein to mean serving as an example, instance, or illustration. Any aspect or design described herein as “example” is not necessarily to be construed as preferred or advantageous over other aspects or designs. Rather, use of the word “example” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an implementation” or “one implementation” throughout is not intended to mean the same embodiment or implementation unless described as such.

Implementations the systems, algorithms, methods, instructions, etc., described herein can be realized in hardware, software, or any combination thereof. The hardware can include, for example, computers, intellectual property (IP) cores, application-specific integrated circuits (ASICs), programmable logic arrays, optical processors, programmable logic controllers, microcode, microcontrollers, servers, microprocessors, digital signal processors, or any other suitable circuit. In the claims, the term “processor” should be understood as encompassing any of the foregoing hardware, either singly or in combination. The terms “signal” and “data” are used interchangeably.

As used herein, the term module can include a packaged functional hardware unit designed for use with other components, a set of instructions executable by a controller (e.g., a processor executing software or firmware), processing circuitry configured to perform a particular function, and a self-contained hardware or software component that interfaces with a larger system. For example, a module can include an application specific integrated circuit (ASIC), a Field Programmable Gate Array (FPGA), a circuit, digital logic circuit, an analog circuit, a combination of discrete circuits, gates, and other types of hardware or combination thereof. In other embodiments, a module can include memory that stores instructions executable by a controller to implement a feature of the module.

Further, in one aspect, for example, systems described herein can be implemented using a general-purpose computer or general-purpose processor with a computer program that, when executed, carries out any of the respective methods, algorithms, and/or instructions described herein. In addition, or alternatively, for example, a special purpose computer/processor can be utilized which can contain other hardware for carrying out any of the methods, algorithms, or instructions described herein.

Further, all or a portion of implementations of the present disclosure can take the form of a computer program product accessible from, for example, a computer-usable or computer-readable medium. A computer-usable or computer-readable medium can be any device that can, for example, tangibly contain, store, communicate, or transport the program for use by or in connection with any processor. The medium can be, for example, an electronic, magnetic, optical, electromagnetic, or a semiconductor device. Other suitable mediums are also available.

The above-described embodiments, implementations, and aspects have been described in order to allow easy understanding of the present invention and do not limit the present invention. On the contrary, the invention is intended to cover various modifications and equivalent arrangements included within the scope of the appended claims, which scope is to be accorded the broadest interpretation to encompass all such modifications and equivalent structure as is permitted under the law.

As used herein, plural instances may be provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and may fall within a scope of various embodiments of the present disclosure. In general, structures and functionality presented as separate resources in the example configurations may be implemented as a combined structure or resource. Similarly, structures and functionality presented as a single resource may be implemented as separate resources.

These and other variations, modifications, additions, and improvements fall within a scope of embodiments of the present disclosure as represented by the appended claims. The specification and drawings are, accordingly, to be regarded in an illustrative rather than a restrictive sense.

What is claimed is:

1. A system comprising:
a processor; and
a memory component having medical data records and instructions stored thereon, when executed by the processor, causes the processor to:
compute a compliance score from an ideal treatment plan and an actual treatment, the actual treatment being stored in the medical data records;
determine whether the compliance score is greater than a compliance threshold;
in response to a determination that the compliance score is greater than the compliance threshold, store a pass flag with a data record of the actual treatment; and
in response to a determination that the compliance score is less than the compliance threshold:
store a fail flag with the data record of the actual treatment; and
control a graphical user display to indicate the data record includes the fail flag.

2. The system of claim 1, wherein the instructions further cause the processor to, in response to storing a pass flag with the data record, control the graphical user display to indicate the data record includes the pass flag.

3. The system of claim 1, wherein the instructions further cause the processor to compute costs for the ideal treatment plan based on a geographic area and costs for the actual treatment received for a same injury to an individual.

4. The system of claim 1, wherein the medical data records include insurance claim records for an individual.

5. The system of claim 1, wherein the instructions further cause the processor to control the graphical user display by limiting the graphical user display based on geographic area.

6. The system of claim 1, wherein the instructions further cause the processor to trigger remedial events when the fail flag is generated or present in the data record.

7. The system of claim 1, wherein the instructions further cause the processor to trigger decision support events when the fail flag is generated or present in the data record.

8. A method comprising:
receiving medical data records;
computing a compliance score from an ideal treatment plan and an actual treatment, the actual treatment stored in medical data records;
determining whether the compliance score is greater than a compliance threshold;
in response to a determination that the compliance score is greater than the compliance threshold, storing a pass flag with a data record of the actual treatment; and
in response to a determination that the compliance score is less than the compliance threshold:
storing a fail flag with the data record of the actual treatment; and
controlling a graphical user display to indicate the data record includes the fail flag.

9. The method of claim 8, further comprising, in response to storing a pass flag with the data record, controlling the graphical user display to indicate the data record includes the pass flag.

10. The method of claim 8, further comprising computing costs for the ideal treatment plan based on a geographic area and costs for the actual treatment received for a same injury to an individual.

11. The method of claim 8, wherein the medical data records include insurance claim records for an individual.

12. The method of claim 8, further comprising controlling the graphical user display by limiting the graphical user display based on geographic area.

13. The method of claim 8, further comprising triggering remedial events when the fail flag is generated or present in the data record.

14. The method of claim 8, further comprising triggering decision support events when the fail flag is generated or present in the data record.

15. A system comprising:
a processor; and
a memory component having medical data records and instructions stored thereon, when executed by the processor, causes the processor to:
receive an ideal treatment plan corresponding to an injury of an individual;
identify an actual treatment in the medical data records corresponding to the ideal treatment plan;
generate, a compliance score based on the ideal treatment plan and the actual treatment;
determine whether the compliance score is greater than a compliance threshold;
in response to a determination that the compliance score is greater than the compliance threshold:
store a pass flag with a data record of the actual treatment; and
control a graphical user display to indicate the data record includes the pass flag;
in response to a determination that the compliance score is less than the compliance threshold:
store a fail flag with the data record of the actual treatment;
control the graphical user display to indicate the data record includes the fail flag; and
trigger intervention in response to the fail flag being stored in the data record of the actual treatment; and
in response to intervention being triggered:
generate, using machine learning, a calibrated compliance threshold corresponding to the compliance score;
in response to a determination that the compliance score is less than the calibrated compliance threshold, continue the triggered intervention.

16. The system of claim 15, wherein the instructions further cause the processor to compute costs for the ideal treatment plan based on a geographic area and costs for the actual treatment received for a same injury to the individual.

17. The system of claim 15, wherein the medical data records include insurance claim records for the individual.

18. The system of claim 15, wherein the instructions further cause the processor to in response to a determination that the compliance score is greater than the calibrated compliance threshold, discontinue the triggered intervention.

19. The system of claim 15, wherein the instructions further cause the processor to trigger remedial events in response to intervention being triggered.

20. The system of claim 15, wherein the instructions further cause the processor to trigger decision support events in response to intervention being triggered.
